(19) United States
(12) Patent Application Publication
(10) Pub. No.: US 2011/0280936 A1
(43) Pub. Date: Nov. 17, 2011

(54) Self Breaking Tablets

(75) Inventors: Siva Rama Krishna Nutalapati, Princeton, NJ (US); Rakeshkumar Khushalbhai Lad, Marlton, NJ (US)

(73) Assignee: Aptapharma, Inc., Pennsauken, NJ (US)

(21) Appl. No.: 13/104,218

(22) Filed: May 10, 2011

Related U.S. Application Data
(60) Provisional application No. 61/345,215, filed on May 17, 2010.

(51) Int. Cl. A61K 9/48; A61K 9/28

(52) U.S. Cl. 424/452; 424/465; 427/2.14

(57) ABSTRACT

Self-breaking tablet and capsule formulations with a similar in vitro drug release profile for whole tablet and when broken and/or a bioequivalent drug release profile when taken whole or when broken are provided. Methods for production of these formulations and tablets and their administration are also provided.

This patent application claims the benefit of priority from U.S. Provisional Application Ser. No. 61/345,215, filed May, 2010, the teachings of which are herein incorporated by reference in their entirety.

### FIELD OF THE INVENTION

The present invention provides oral formulations of tablets which self break into a plurality of drug containing units for easier swallowing. Formulations of the present invention provide a similar in vitro drug release profile for the whole tablet and when broken and/or a bioequivalent drug release profile when taken whole or when broken into the plurality of drug containing. Methods for production and use of these oral formulations are also provided.

### BACKGROUND OF THE INVENTION

People often need to break or sprinkle their medications if they cannot swallow the entire tablet or capsule. Often the contents of a capsule is sprinkled into a fluid, swirled and then swallowed. For a medicine to be approved by the Food and Drug Administration for administration via this manner, the drug release profile upon sprinkling must be bioequivalent to the drug release profile of the whole tablet or capsule.

For many tablets, particularly coated controlled, modified or delayed release prescription medication tablets, the drug release profile is altered or compromised upon breaking or splitting. Generally, single unit coated tablets cannot be broken into small pieces without compromising the coating. Such compromises result in differences in drug release profile rendering breaking or sprinkling of the tablet or capsule unacceptable.

Multiparticulate formulations are currently the only FDA approved formulations that can be taken whole or sprinkled.

The American Medical Society and American Pharmacists Association recommends against splitting tablets that are modified release, combination products, unscored, film coated, friable or dose critical (Noviasky et al. The Journal of Family Practice, jfponline with the extension .com/Pages.asp?AID=4326 of the world wide web).

### SUMMARY OF THE INVENTION

The present invention relates to oral formulations comprising a core tablet which self breaks via a continuous quick disintegrating layer into a plurality of drug containing units for easier swallowing. Unlike multiparticulate formulations which comprise a plurality of drug containing particles prepared separately and then compressed into a single core tablet, in the formulations of the present invention, the core tablet is formed by multilayer tablet compression wherein a layer of drug containing units is compressed with or to a layer of continuous quick distintegrating material. Each drug containing unit in the layer of drug containing units is multilayered comprising at least one drug containing layer and one plug layer. The plug layers of the drug containing units are joined by the continuous quick disintegrating layer compressed thereto to form the core tablet. The core tablet may be optionally coated with a functional coating. The core tablet may be further optionally coated with a subcoating between the core tablet and the functional coating. When the tablet of the present invention comes in contact with fluid, the quick disintegrating layer disintegrates leaving the individual drug containing units dispersed in the fluid.

Tablets of the present invention have a similar in vitro drug release profile for the whole tablet and when disintegrated into a plurality of drug containing units and/or a similar bioequivalent drug release profile when taken whole or when taken as a plurality of drug containing units.

In one embodiment of the present invention, one or more tablets of the present invention are encapsulated in a capsule.

In another embodiment, the tablet of the present invention is compressed into a second tablet thereby forming a tablet in a tablet.

As the tablet of the present invention disintegrates into its plurality of drug containing units upon addition of the tablet to a fluid such as water or juice, oral formulations of the present invention are useful in patients who cannot swallow entire tablets or capsules.

Another aspect of the present invention relates to a method for producing tablets comprising a layer of drug containing units which self breaks upon addition to a fluid into the drug containing units for easier swallowing. The method comprises compressing the quick disintegrating core tablet comprising a layer of multiple drug containing units and optionally applying the coating.

Another aspect of the present invention relates to a method for orally administering a drug to a patient which comprises adding a tablet of the present invention to a fluid so that the tablet self-breaks into a plurality of drug containing units and administering the fluid to the patient.

### BRIEF DESCRIPTION OF THE FIGURES

FIG. 1 is a diagram of an exemplary tablet of the present invention with a plurality of drug containing units which self breaks into the drug containing units upon addition of the tablet to a fluid.

FIG. 2 is a diagram of a closer view of a core tablet with the drug containing unit and quick disintegrating layer.

FIG. 3 shows diagrams of cross-sections of two embodiments of dies used to produce a tablet of the present invention. The shape of the die and the number of holes per die can be modified as required in accordance with the number of drug containing units to be incorporated in the core tablet.

### DETAILED DESCRIPTION OF THE INVENTION

The present invention provides new formulations for orally administered drugs which self break into a plurality of drug containing units upon addition to a fluid. The formulations of the present invention exhibit a similar in vitro drug release profile for whole tablet and when broken into the plurality of drug containing units and/or similar bioequivalent drug release profiles when taken whole or when broken into the plurality of drug containing units.

Formulations of the present invention can be used with any orally administered drug which can be compressed into a tablet.

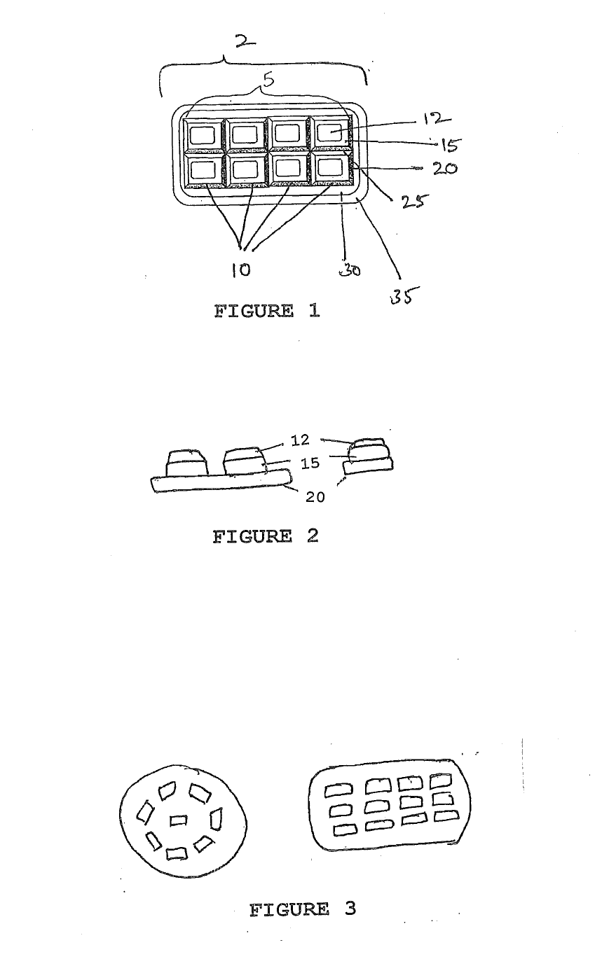

One embodiment of a tablet of the present invention with a layer of drug containing units which self breaks into the drug containing units is depicted in FIG. 1.

As shown in FIG. 1, a tablet 2 of the present invention comprises a core tablet 5 comprising a layer of drug containing units 10. In one embodiment the core tablet comprises a layer with four or more drug containing units. A closer view of the drug containing unit is provided in FIG. 2. Each drug containing unit 10 comprises a drug containing layer 12 and a plug layer 15. The layer of drug containing units is compressed into the core tablet 5 with or to a continuous quick disintegrating layer 20 adjacent to the plug layer 15 of each drug containing unit 10. See FIG. 2. The core tablet 5 may be optionally coated with a functional coating 35. The core tablet 5 may be further optionally coated with a subcoating 30 between the core tablet 5 and the functional coating 35.

In one embodiment of the present invention, wherein tablets are compressed using a multilayer tablet press fitted with a caplet shaped tooling, grid lines or scores 25 are carved out in the layer of drug containing units.

The drug containing unit may comprise a single orally administered drug or multiple orally administered drugs. Each drug containing unit may comprise the same amount of drug or drugs. Alternatively, each drug containing unit may contain different amounts of drug or drugs and/or different drugs when more than one drug is being administered.

The drug containing unit may comprise more than one drug and/or may exhibit different drug release profiles including immediate release, delayed release, controlled release and/or modified release.

Any orally active agent can be included as a drug in the drug containing units. Examples include, but are not limited to, alpha-2 adrenergic agents, analgesics, angiotensin-converting enzyme (ACE) inhibitors, antianxiety agents, antiarrhythmics, antibacterials, antibiotics, anticoagulants, anticonvulsants, antidepressants, antidiabetics, antiemetics, antiepileptics, antifungals, antihelminthics, antihistamines, antihyperlipidemics, antihypertensives, antiinfectives, antimalarials, antimicrobials, antimigraine agents, antimuscarinic agents, antineoplastic agents, antiprotozoals, antipsychotics, antispasmodics, antiretroviral agents, antivirals, attention-deficit hyperactivity disorder (ADHD) agents, β-blockers, calcium channel blockers, chemotherapeutic agents, cholinesterase inhibitors, Cox-2 inhibitors, decongestants, diuretics, histamine-2 receptor antagonists, hypnotics, hypoglycemic agents, hypotensive agents, immunosuppresants, lipotropics, neuroleptics, opioid analgesics, peripheral vasodilators/vasoconstrictors, proton pump inhibitors, sedatives, serotonin receptor agonists, sympathomimetics as well as pharmaceutically acceptable salts, solvates, hydrates, stereoisomers (racemates, individual enantiomers or diastereomers, or any combination thereof), or polymorphs thereof, or pharmaceutically acceptable combinations comprising at least one of the foregoing active agents, and the like.

The core tablet containing the layer of drug containing units is prepared first. Each drug containing unit comprises at least one drug containing layer and one plug layer. The plug layer of all drug containing units is compressed with or to the continuous quick disintegrating layer of the tablet. When this tablet comes in contact with a fluid, the quick disintegrating layer disintegrates immediately leaving the individual drug containing units dispersed in the fluid.

As will be understood by the skilled artisan upon reading this disclosure, drug containing units of the present invention may comprise additional drug containing layers as well as additional plug layers in between.

In one embodiment, the drug containing layer of the drug containing units of the tablets of the present invention is prepared by direct compression of a mixture of the drug or drugs with a suitable carrier or excipient, such as carbohydrate, cellulose or protein fillers, such as sugars, including lactose, sucrose, mannitol, or sorbitol; starch from corn, wheat, rice, potato, or other plants; cellulose, such as methyl cellulose, hydroxypropylmethyl cellulose, sodium carboxymethylcellulose, or microcrystalline cellulose; gums including arabic and tragacanth; proteins such as gelatin and collagen; inorganics, such as kaolin, calcium carbonate, dicalcium phosphate, sodium chloride; magnesium carbonate; magnesium oxide; and other agents such as acacia and alginic acid.

Agents that facilitate disintegration and/or solubilization can be added to the drug containing layer. Examples include, but are not limited to cross-linked polyvinyl pyrrolidone, sodium starch glycolate, Croscarmellose Sodium, alginic acid, or a salt thereof, such as sodium alginate, microcrystalline cellulose and corn, starch.

Tablet binders can also be added to the drug containing layer. Examples of binders include, but are not limited to, acacia, methylcellulose, sodium carboxymethylcellulose, polyvinylpyrrolidone (POVIDONE), hydroxypropyl cellulose, hydroxypropyl methylcellulose, sucrose, starch and ethylcellulose.

Lubricants can also be added to the drug containing layer. Examples include, but are not limited to, magnesium stearates, stearic acid, sodium stearyl fumerate, talc, waxes, oils, silicon dioxide and colloidal silica.

Drug containing layers of the drug containing units of the tablets are formulated, for example, by preparing a powder mixture of drug or drugs by dry blending or granulating or slugging, adding a lubricant and disintegrant and pressing the mixture into tablet layers.

A modified release drug containing unit of the tablet can be prepared by incorporating release retarding excipients into the above-described formulation for the immediate release drug containing layer, and either completely omitting or reducing the amount of disintegrants.

Examples of release retarding excipients include, but are not limited to hydrophilic polymers such as hydroxypropylmethylcellulose, hydroxymethylcellulose, hydroxypropylcellulose and hydroxyethylcellulose, and which swell in contact with aqueous liquids, and control release of the drug by diffusion through the swollen polymer network.

Examples of other release retarding excipients include, but are not limited to, waxes such as carnauba wax, bees wax stearic acid and gums such as acacia, acrylic polymers, shellac, zein, polyvinylpyrrolidine including crosslinked polyvinylpyrrolidinone, vinyl acetate copolymers, polyethylene oxides, polyvinyl alcohols, and combinations comprising at least one of the foregoing materials.

The modified release drug containing units of the tablet are formulated, for example, by preparing the powder mixture of drug or drugs by dry blending or granulating or slugging, adding a lubricant and release retarding excipients and pressing the mixture into a drug containing layer of the drug containing unit.

Each drug containing unit further comprises a plug layer connecting the drug containing units to the quick disintegrating layer. Preferably, the plug material connecting the drug containing units to the quick disintegrating layer is inert, soluble or insoluble and preferably impermeable to drug in the drug containing layer of the drug containing units regardless of thickness so that self breaking of the tablet into the drug containing units upon addition of the tablet to a fluid has no impact on drug release. In one embodiment, this plug layer comprises no drug or drug in an amount which does not significantly modify the in vitro drug release profile and/or bioequivalence and/or functionality of a functional coating or film on the tablet upon breaking. As will be understood by the skilled artisan upon reading this disclosure, however, drug in an amount which does modify bioequivalence and/or functionality may be included in this material and such inclusion does not circumvent this invention.

Exemplary biocompatible materials for use in the plug layer include, but not limited to, waxes, polymers, gums and other pharmaceutically acceptable excipients either alone or in combination.

Exemplary wax excipients include, but are not limited to, wax and wax-like excipients such as carnauba wax, vegetable wax, fruit wax, microcrystalline wax, bees wax (white or bleached, and yellow), hydrocarbon wax, paraffin wax, cetyl esters wax or a combination comprising at least one of the foregoing waxes. Other suitable wax excipients include, for example, fatty alcohols (such as lauryl, myristyl, stearyl, cetyl or specifically cetostearyl alcohol), hydrogenated vegetable oil, hydrogenated castor oil, fatty acids such as stearic acid, fatty acid esters including fatty acid glycerides (mono-, di-, and tri-glycerides), polyethylene glycol (PEG) having a molecular weight of greater than about 3000 number average molecular weight, Mₙ (e.g. PEG 3350, PEG 4000, PEG 4600, PEG 6000, and PEG 8000), or a combination comprising at least one of the foregoing.

Exemplary polymer excipients include, for example acrylic polymers, alkylcelluloses including substituted alkylcelluloses, shellac, zein, polyvinylpyrrolidine including crosslinked polyvinylpyrrolidinone, vinyl acetate copolymers, polyethylene oxides, polyvinyl alcohols, and combinations comprising at least one of the foregoing materials.

Suitable acrylic polymers that can be used in the plug layer include, but are not limited to, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, or a combination comprising at least one of the foregoing polymers.

Suitable alkylcelluloses and substituted alkyl celluloses can be used in the plug layer include, but are not limited to, methyl cellulose, ethylcellulose, hydroxy or carboxy substituted alkyl celluloses (e.g., hydroxyl propylcellulose, crosslinked hydroxypropylcellulose, carboxymethylcellulose, crosslinked sodium carboxymethylcellulose), hydroxy substituted alkyl-alkyl celluloses (e.g., hydroxypropylmethylcellulose), or a combination comprising at least one of the foregoing.

Exemplary additional pharmaceutically acceptable excipients for use in the plug layer include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, magnesium aluminum silicate (Veegum), and larch arabogalactan); alginates; polyethylene oxide; inorganic calcium salts; silicic acid; and combinations thereof.

Fillers, tablet binders, pH modifiers and lubricants, including the aforementioned, can be used in the plug layer singly or in combination.

In one embodiment, the plugs of the core tablet of the present invention are formulated, for example, by preparing a powder mixture of plug material by dry blending or granulating or slugging, adding a lubricant and pressing into tablet layers.

The quick disintegrating layer of the tablet of the present invention is prepared by direct compression of suitable carriers or excipients, such as carbohydrate, cellulose or protein fillers, such as sugars, including lactose, sucrose, mannitol, or sorbitol; starch from corn, wheat, rice, potato, or other plants; cellulose, such as methyl cellulose, hydroxypropylmethyl cellulose, sodium carboxymethylcellulose, or microcrystalline cellulose; gums including arabic and tragacanth; proteins such as gelatin and collagen; inorganics, such as kaolin, calcium carbonate, dicalcium phosphate, sodium chloride; magnesium carbonate; magnesium oxide; and other agents such as acacia and alginic acid.

Agents that further facilitate disintegration and/or solubilization such as superdisintegrants are added to this quick disintegrating layer. Examples include, but are not limited to cross-linked polyvinyl pyrrolidone, sodium starch glycolate, Croscarmellose Sodium, alginic acid, or a salt thereof, such as sodium alginate, microcrystalline cellulose and corn starch.

Tablet binders can also be used in the quick disintegrating layer of the tablet. Examples include, but are not limited to, acacia, methylcellulose, sodium carboxymethylcellulose, polyvinylpyrrolidone (POVIDONE), hydroxypropyl cellulose, hydroxypropyl methylcellulose, sucrose, starch and ethylcellulose.

Lubricants can also be used in the quick disintegrating layer of the tablet. Examples include, but are not limited to, magnesium stearates, stearic acid, sodium stearyl fumerate, talc, waxes, oils, silicon dioxide and colloidal silica.

A drug or drugs for immediate release upon contact of the tablet with an aqueous media may also be included in the quick disintegrating layer.

Alternatively, or in addition, agents that improve patient compliance such as flavoring agents, sweeteners, effervescent agents, coloring agents and such can also be added to the quick disintegrating layer.

Materials that are incompatible to the functional coatings can be added to the quick disintegrating layer to facilitate the disintegration. For example Eudragit E100 can be added to the quick disintegrating layer when the tablets are coated with Eudragit L30D55. Since Eudragit E100 and Eudragit L30D55 are incompatible to each other, addition of Eudragit E100 causes premature release of the tablet contents when coated with Eudragit L30D55. Similarly addition of basic compounds which are incompatible to Eudragit coatings such as but not limited to Sodium Phosphate, Dibasic Potassium Carbonate, Tri basic Potassium Phosphate, Sodium Hydroxide etc. can be added to the quick disintegrating layer to facilitate the disintegration.

The quick disintegrating layer or layers of the tablets are formulated, for example, by preparing a powder mixture of the excipients with or without the drug(s), by dry blending or granulating or slugging, adding a lubricant and additional disintegrant and pressing the mixture into tablet layers.

The core tablet can then be coated with one or more functional coatings or films. By “functional coating or film” it is meant a coating that modifies the release properties of the total formulation. Examples of such coatings or films include, but are not limited to, controlled release, delayed release, modified release, enteric coating, pH dependent coatings, pH independent coatings, and any combinations thereof.

The functional coating material can be in the form of a film coating comprising a solution or dispersion or a compressible powder mixture of a hydrophilic and/or hydrophobic polymer. Solvents used for application of the functional coating include pharmaceutically acceptable solvents, such as water, methanol, ethanol, isopropanol, acetone, methylene chloride, and a combination comprising at least one of the foregoing solvents.

Exemplary functional coating materials include film forming polymers such as acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, an alkylcellulose including methylcellulose or ethylcellulose, a hydroxyalkylcellulose such as hydroxymethylcellulose, hydroxyethylcellulose, hydroxypropylcellulose and hydroxybutylcellulose, a hydroxyalkyl alkylcellulose such as hydroxyethyl methylcellulose and hydroxypropyl methylcellulose, a carboxyalkylcellulose such as carboxymethylcellulose, an alkali metal salt of carboxyalkylcelluloses such as sodium carboxymethylcellulose, a carboxyalkyl alkylcellulose such as carboxymethyl ethylcellulose, a carboxyalkylcellulose ester, a starch, a pectin such as sodium carboxymethylamylopectine, a chitin derivate such as chitosan, a polysaccharide such as alginic acid, alkali metal and ammonium salts thereof, a carrageenan, a galactomannan, traganth, agar-agar, gum arabicum, guar gum and xanthan gum, a polyacrylic acid and the salts thereof, a polyvinylalcohol, a polyvinylpyrrolidone, a copolymer of polyvinylpyrrolidone with vinyl acetate, a polyalkylene oxide such as polyethylene oxide and polypropylene oxide and a copolymer of ethylene oxide and propylene oxide, or a combination comprising at least one of the foregoing.

The functional coating may optionally comprise a plasticizer, an additional film-former, a pore former, a release modifier or a combination comprising at least one of the foregoing.

The formulations of the present invention can optionally further comprise a subcoating or non-functional coating. By “non-functional coating” it is meant a coating that does not significantly modify the release properties of the total formulation, for example, a cosmetic coating or an interlayer coating used to separate a functional coating from other components of the formulation. A non-functional coating can have some impact on the release of the active agent due to the initial dissolution, hydration, perforation of the coating, etc., but is not considered to cause significant deviation in release from the non-subcoated composition.

Exemplary subcoating materials include, but are not limited to, film forming polymers like hydroxyalkyl celluloses such as hydroxymethyl cellulose, hydroxyethyl cellulose, hydroxypropyl cellulose and hydroxybutylcellulose, hydroxyalkyl alkylcelluloses such as hydroxyethyl methylcellulose and hydroxypropyl methylcellulose, polyvinylalcohols, polyvinylpyrrolidones, copolymers of polyvinylpyrrolidone with vinyl acetate, and combinations thereof.

For the embodiment of the present invention wherein the tablet has a bioequivalent drug release profile when taken whole or when broken, the drug containing units of the tablet of the present invention are sized so that upon self breaking of the tablet upon addition to a fluid, the drug containing units of the tablet can be more easily swallowed. Breakage of the tablet into the drug containing units does not result in changes in the drug release profiles of the tablet either in vivo or in vitro as compared to the tablet as a whole.

For capsule formulations of the present invention, one or more of the tablets are encapsulated within a capsule. Prior to administration to a patient with difficulty swallowing, the capsule is opened and the tablet or tablets are added to a fluid so that that the tablet or tablets self-breaks into its drug containing units.

Also provided in the present invention are methods for producing tablet and capsule formulations which exhibit bioequivalent drug release profiles when taken whole and when broken.

In one embodiment, the method comprises compressing into a core tablet a layer of drug containing units, each unit comprising a drug containing layer separated by a plug layer. The plug layers of all the drug containing units are joined to the continuous quick disintegrating layer of the core tablet. When this tablet comes in contact with a fluid, the quick disintegrating layer disintegrates immediately leaving the individual drug containing units dispersed in the fluid. In one embodiment, the core tablet is selectively coated with one or more functional films or coatings. In this embodiment, the core tablet may be optionally coated with a subcoating prior to coating with the functional film or coating. In another embodiment, the core tablet is compressed into a tablet. In another embodiment, the core tablet is encapsulated in a capsule. In yet another embodiment, the core tablet is selectively coated with a functional film or coating and then either compressed into a second tablet or encapsulated into a capsule. In this embodiment, the core tablet may be optionally coated with a subcoating prior to coating with the functional film or coating.

Tablets can be compressed using a multilayer tablet press. In one embodiment, the multilayer tablet is prepared by compressing into a layer of drug containing units at least one drug layer and a plug layer, and further compressing a quick disintegrating layer to the plug layers of the drug containing units to form the core tablet. Where more than one drug needs to be included, both drug layers are compressed first, then plug layer is compressed and finally the quick disintegrating layer is compressed.

The core tablet is compressed using tooling known in the art or making necessary modifications to the tooling.

In one embodiment, a lower punch or upper punch and/or the die is modified as shown in FIG. 3 to include a plurality of holes for production of the layer of drug containing units. In one embodiment, a multitip lower punch is used in combination with a flat face upper punch. The tablets are then compressed using a multilayer tablet press. First, the drug containing layer of the layer of drug containing units is compressed inside the die using the multitip lower punch. Then the plug layer is compressed inside the die and finally the continuous quick disintegrating layer is compressed outside of the die. As will be understood by the skilled artisan upon reading this disclosure, alternative combinations such as, but not limited to, a multitip lower punch in combination with a multitip upper punch or a flat face lower punch in combination with a multitip upper punch can also be used.

In another embodiment, the tablets are compressed using a multilayer tablet press fitted with a caplet shaped tooling. First, the drug containing layer is compressed, then the plug layer is compressed and finally the continuous quick disintegrating layer is compressed. Once the multilayer tablet is punched, grid lines/scores are carved out in the drug and plug layers.

The compressed tablets can be in any shape such as but not limited to round, caplet, oval and modified oval.

The core tablet can then be coated with a functional coating with an optional sub coating between the core tablet and the functional coating. The coating can be applied to the entire core tablet or to a portion of the core tablet leaving at least one surface of the quick disintegrating layer.

The coating can be applied to the core tablets of the invention either by using the traditional coating methods known in the art such as, but not limited, to pan coating, and fluid bed coating or by using special coating techniques. The traditional coating methods allow coating the tablets entirely or wholly. Partial coating can be applied using special coating techniques.

When the whole tablet is coated with a functional coating, a pore former must to be incorporated into the coating so that the quick disintegrating layer can disintegrate immediately leaving the individual drug containing units dispersed in fluid.

When one cannot incorporate the pore former into the functional coating as is the case with enteric coatings, then the coating needs to be eliminated from at least a portion of the tablet either completely or partially to expose the quick disintegrating layer. The coating can be removed either by laser drilling or mechanical drilling or sanding or similar methods known in the art.

In the event the tablet is partially coated where no coating is applied to at least a portion of the quick disintegrating layer, when this tablet comes in contact with fluid the quick disintegrating layer disintegrates immediately leaving the individual drug containing units dispersed in fluid.

Partial coating can be applied using special coating techniques such as, but not limited, to dip coating, electrostatic coating and controlled spraying.

In dip coating, coating is applied to the core tablets by dipping them into the coating liquid. The wet tablets are dried in a conventional manner in a coating pan or by special means such as passing them through a drying chamber on a conveyor belt or applying dry air or nitrogen. Alternative dipping and drying steps may be repeated several times to obtain the desired coating. Electrostatic coating is an efficient method of applying coating to conductive, substrates. A strong electrostatic charge is applied to the substrate. The coating material containing conductive ionic species of opposite charge is sprayed onto the charged substrate. In controlled spraying, the coating process is carried out by controlled spraying of the coating solution or suspension onto the preferred surface of the core tablet. The wet tablets are dried in a conventional manner in, for example, a coating pan or by special means such as passing them through a drying chamber on a conveyor belt or applying dry air or nitrogen. Alternative spraying and drying steps may be repeated several times to obtain the desired coating. In all these techniques the composition and consistency of the coating solution or suspension is critical.

These special coating techniques also bring the advantage of selective coating. If the core tablet has, for example, six drug containing units, they can be selectively coated to manipulate the required release profile. In one embodiment, of these six drug containing units, two units can be left without coating to give immediate drug release, two units can be coated with one type of polymer, for example, Eudragit L30D55 which dissolves at pH 5.5 and above, and the remaining two units can be coated with a different type of polymer, for example, Eudragit S100 which dissolves at pH 7.2 and above.

With the help of selective coating techniques, any release profile, such as, but not limited to immediate release, sustained release, delayed release, pH dependent release, pH independent release, and/or pulsatile release can be achieved.

These tablets, either coated or uncoated can optionally be further compressed into a second tablet as a tablet in a tablet form to improve the appearance, ease of swallowing and/or palatability.

The tablets and capsules of the present invention are useful for oral administration of drugs to patients with difficulty in swallowing whole tablets or capsules. For this use, the above described tablet self-breaks into a plurality of drug containing units upon addition to a fluid. The fluid with the drug containing units is then administered orally to the patient. For capsule formulation, the tablet is first removed from its capsule and then added to the fluid wherein it self-breaks into the plurality of drug containing units. Again, the fluid containing the drug containing units is orally administered to the patient.

The following nonlimiting examples are provided to further illustrate the present invention.

### EXAMPLES

### Example 1

### Preparation of Extended Release Multilayer Tablets of Venlafaxine Hydrochloride with 25% Loading Dose

The drug containing layer contained Venlafaxine hydrochloride (37.5 mg), microcrystalline cellulose (21.5 mg), hypromellose (40.00 mg), and magnesium stearate (3.00 mg).

The plug contained carnauba wax (64 mg), dibasic calcium phosphate (25 mg), stearic acid (10 mg) and magnesium stearate (1 mg).

The quick disintegrating layer contained microcrystalline cellulose (35 mg), lactose anhydrous (45 mg), hydroxypropyl cellulose (4 mg), croscarmellose sodium (15 mg), and magnesium stearate (1 mg).

The subcoating contained hydroxypropyl methyl cellulose (12.50 mg/tablet), polyethylene glycol 400 (2.50 mg/tablet) and ethanol which was removed during processing.

The extended release coating contained Ethyl cellulose (30.90 mg/tablet), hydroxypropyl methyl cellulose (20.60 mg/tablet) and ethanol which was removed during processing.

The drug containing layer was prepared as follows:

Venlafaxine hydrochloride was dry blended with all the ingredients except magnesium stearate and granulated with purified water. The granulate was dried and milled through a suitable screen. Magnesium stearate was screened and then added to the milled granules. The mixture was then blended for about 2 minutes.

The plug was prepared as follows:

Carnauba wax and dicalcium phosphate were mixed in a collette and granulated with a solution of stearic acid in ethyl alcohol. The granulate was then dried, milled and transferred to a blender. Magnesium stearate was screened and then added to the blender. The mixture was then blended for another 2 minutes.

The quick disintegrating layer was prepared as follows:

All the ingredients except magnesium stearate were dry blended for five minutes in a blender. Magnesium stearate was screened and then added to the blender. The mixture was then blended for another 2 minutes.

The blends were then compressed into a multi-layer core tablet in the following sequence: drug containing layer, plug and quick disintegrating layer of 100 mg each using a multi-layer tablet press.

Scores are carved out in a grid fashion into the drug and plug layers of the tablet leaving the quick disintegrating layer intact, so that the plug layers of all drug containing units are joined to the continuous quick disintegrating layer of the tablet. After scores are made, the tablet will have eight (8) drug containing units connected to the quick disintegrating layer. Core tablets were then subcoated.

The subcoating was prepared by dissolving hydroxypropyl methyl cellulose and polyethylene glycol 400 in ethanol. Coating is applied to the tablet cores by dipping them into the coating liquid. The wet tablets are dried by passing through a drying chamber on a conveyor belt. Alternative dipping and drying steps may be repeated several times to obtain the desired coating.

The extended release coating was prepared as follows: In a container ethanol was mixed with ethyl cellulose and hydroxypropyl methyl cellulose using mixer until the both were completely dissolved. Coating is selectively applied to only six (6) of the eight drug containing units of the sub coated tablets by dipping them into the coating liquid. The other two drug containing units were protected from being coated by using a specially designed tablet holder. The wet tablets are dried by passing through a drying chamber on a conveyor belt. Alternative dipping and drying steps may be repeated several times to obtain the desired coating.

### Example 2

### Preparation of Enteric-Coated Multilayer Tablets of Omeprazole

The drug containing layer contained Omeprazole magnesium (22.45 mg/tablet), microcrystalline cellulose (64 mg/tablet), lactose anhydrous (190.55 mg/tablet), hydroxypropyl cellulose (10.00 mg/tablet), croscarmellose sodium (10.00 mg/tablet), and magnesium stearate (3.00 mg/tablet).

The plug contained carnauba wax (128.00 mg/tablet), dibasic calcium phosphate (50.00 mg/tablet), stearic acid (20.00 mg/tablet) and magnesium stearate (2.00 mg/tablet).

The quick disintegrating layer contained microcrystalline cellulose (35 mg), lactose anhydrous (45 mg), hydroxypropyl cellulose (4 mg), croscarmellose sodium (15 mg), and magnesium stearate (1 mg).

The subcoating contained hydroxypropyl methyl cellulose (15.00 mg/tablet), Polyethylene glycol 400 (3.00 mg/tablet) and ethanol which was removed during processing.

The enteric coating contained Eudragit L100 (24.32 mg/tablet), triethyl citrate (2.66 mg/tablet), talc (14.62 mg/tablet) and ethanol which was removed during processing.

The drug containing layer was prepared as follows:

Omeprazole magnesium was dry blended with all the ingredients except magnesium stearate for five minutes in a blender. Magnesium stearate was screened and then added to the blender. The mixture was then blended for another 2 minutes.

The plug was prepared as follows:

Carnauba wax and dicalcium phosphate were mixed in a collette and granulated with a solution of stearic acid in ethyl alcohol. The granulate was then dried, milled and transferred to a blender. Magnesium stearate was screened and then added to the blender. The mixture was then blended for another 2 minutes.

The quick disintegrating layer was prepared as follows:

All the ingredients except magnesium stearate were dry blended for five minutes in a blender. Magnesium stearate was screened and then added to the blender. The mixture was then blended for another 2 minutes.

The blends were then compressed into a multi-layer core tablet in the following sequence: drug containing layer (150 mg, plug (100 mg) and quick disintegrating layer (100 mg) using a multi-layer tablet press equipped with a special tooling. First the drug layer is compressed inside the die using the multitip bottom punch. Then the plug layer is compressed inside the die and finally the continuous quick disintegrating layer is compressed outside of the die. Core tablets were then subcoated.

The subcoating was prepared by dissolving hydroxypropyl methyl cellulose and polyethylene glycol 400 in ethanol. Coating is applied to the tablet cores by dipping them into the coating liquid. The wet tablets are dried by passing through a drying chamber on a conveyor belt. Alternative dipping and drying steps may be repeated several times to obtain the desired coating.

The enteric coating was prepared by dissolving Eudragit L100 and triethyl citrate in ethanol in a container using a mixer. Talc was added and the whole suspension was mixed until the talc was evenly dispersed. Coating is applied to the sub coated tablets by dipping them into the coating liquid. The wet tablets are dried by passing through a drying chamber on a conveyor belt. Alternative dipping and drying steps may be repeated several times to obtain the desired coating.

What is claimed is:

1. A core tablet comprising a layer of drug containing units produced by compression, each drug containing unit comprising a drug containing layer and a plug layer, said core tablet further comprising a continuous quick disintegrating layer compressed with the layer of drug containing units which joins the drug containing units to the core tablet via their plug layers.

2. The core tablet of claim 1 wherein the drug containing units comprise a single drug.

3. The core tablet of claim 1 wherein the drug containing units comprise two or more drugs.

4. The core tablet of claim 1 which self-breaks into the drug containing units upon addition of the core tablet to a fluid via disintegration of the continuous quick disintegrating layer.

5. The core tablet of claim 1 wherein the drug containing units comprise more than one drug containing layer and/or more than one plug layer.

6. A tablet comprising the core tablet of claim 1 coated in whole or in part with a functional coating or film.

7. The tablet of claim 6 further comprising a subcoating between the core tablet and the functional coating or film.

8. The tablet of claim 6 wherein the functional coating or film comprises a pore former.

9. The tablet of claim 6 wherein a portion of the continuous quick disintegrating layer is left uncoated.

10. The tablet of claim 6 wherein the drug containing units of the core tablet are selectively coated with different functional coatings.

11. A tablet in tablet form comprising a core tablet of claim 1 further compressed into a second tablet.

12. A tablet in tablet form comprising a tablet of claim 6 further compressed into a second tablet.

13. A capsule comprising one or more core tablets of claim 1 encapsulated within a capsule.

14. A capsule comprising one or more tablets of claim 1 encapsulated within a capsule.

15. A method of administering a drug orally to a subject, said method comprising adding to a fluid the core tablet of claim 1 so that the core tablet self-breaks into the drug containing units and administering the fluid containing the drug containing units orally to the subject.

16. A method of administering a drug orally to a subject, said method comprising adding to a fluid the tablet of claim 6 so that the tablet self-breaks into the drug containing units and administering the fluid containing the drug containing units orally to the subject.

17. A method for producing the core tablet of claim 1, said method comprising:
(a) compressing into a tablet a layer of drug containing units, each drug containing unit comprising a drug containing layer and a plug layer; and
(b) joining the plug layers of all the drug containing units to a continuous quick disintegrating layer to form the core tablet.

18. The method of claim 17 wherein a single drug is compressed into the drug containing layer of the drug containing units.

19. The method of claim 17 wherein two or more drugs are compressed into the drug containing layer of the drug containing units.

20. The method of claim 17 wherein more than one drug containing layer and/or more than one plug layer are compressed into the drug containing units.

21. The method of claim 20 wherein the core tablet is compressed with lower and upper punches and a die modified to include a plurality of holes for production of the layer of drug containing units.

22. The method of claim 20 wherein the lower punch and/or upper punch is a multitip punch and the core tablet is produced by compressing the drug containing layer inside the die via the upper and/or lower multitip punch, compressing the plug layer inside the die via the upper and/or lower multitip punch and compressing the continuous quick disintegrating layer outside of the die.

23. The method of claim 17 wherein the core tablet is compressed with a multilayer tablet press.

24. The method of claim 23 further comprising carving grid lines or scores into the layer of drug containing units.

25. A method for producing a tablet, said method comprising:
(a) producing a core tablet in accordance with the method of claim 14; and
(b) coating the core tablet in whole or in part with a functional coating or film.

26. The method of claim 25 further comprising applying a subcoating between the core tablet and the functional coating or film.

27. The method of claim 25 wherein the functional coating or film comprises a pore former.

28. The method of claim 25 wherein a portion of the continuous quick disintegrating layer of the core tablet is left uncoated with the functional coating or film.

29. The method of claim 25 wherein different functional coatings are selectively applied to the plurality of drug containing units of the core tablet.

30. The method of claim 25 further comprising compressing the tablet into a second tablet to produce a tablet in tablet form.

31. The method of claim 25 further comprising encapsulating one or more of the tablets into a capsule.
